(19) United States
(12) Patent Application Publication
(10) Pub. No.: US 2020/0085931 A1
(43) Pub. Date: Mar. 19, 2020

(54) PROTEIN NANOPARTICLES AND COMBINATION THERAPY FOR CANCER IMMUNOTHERAPY

(71) Applicant: The Regents of the University of California, Oakland, CA (US)

(72) Inventors: Szu-Wen Wang, Irvine, CA (US); Nicholas Molino, Philadelphia, PA (US); Medea Neek, Irvine, CA (US); Edward L. Nelson, Irvine, CA (US); Jo Anne Tucker, Irvine, CA (US)

(21) Appl. No.: 16/570,834

(22) Filed: Sep. 13, 2019

Related U.S. Application Data
(60) Provisional application No. 62/733,331, filed on Sep. 19, 2018.

(51) Int. Cl. A61K 39/00; A61K 39/39; A61P 35/00; A61K 38/17

(52) U.S. Cl. CPC A61K 39/001188; A61K 39/001186; A61K 39/39; A61K 2039/55561; A61K 38/1774; A61K 2039/6031; A61K 2039/572; A61P 35/00

(57) ABSTRACT

Cancer-testis antigens were simultaneously packaged with CpG adjuvant and incorporated into an E2 nanoparticle platform to increase cancer vaccine efficacy. Also described herein is a combination of checkpoint blockade therapy and the nanoparticle vaccine platform to deliver cancer antigens with adjuvant for treatment of tumors and prevention of future tumors. The nanoparticle vaccine platform includes a protein capsule to which are attached adjuvants in the internal hollow cavity and cancer epitopes to the surface. Whereas single-therapies only increase survival, the combined therapy can both increase survival time as well as prevent tumor development in pre-existing tumor conditions by increasing tumor antigen-specific responses (via the nanoparticle vaccines) while simultaneously blocking checkpoints to remove immune suppression (via immune checkpoint inhibition). Furthermore, tumor re-challenge studies show evidence of T cell memory which can prevent tumor development in some individuals.

### CROSS-REFERENCES TO RELATED APPLICATIONS

This application is a non-provisional application and claims benefit of U.S. Provisional Patent Application No. 62/733,331, filed Sep. 19, 2018, the specification of which is incorporated herein in their entirety by reference.

### GOVERNMENT SUPPORT

This invention was made with government support under Grant Nos. R21 EB017995 and P30CA062203, awarded by the National Institutes of Health. The Government has certain rights in the invention.

### FIELD OF THE INVENTION

The present invention relates to cancer treatments, in particular, to protein nanoparticle vaccines and combination therapy with checkpoint inhibitors.

### BACKGROUND OF THE INVENTION

Boosting a patient's immune system by immunotherapy represents a promising approach in cancer treatment, and cancer vaccines in particular can prime the immune system to better recognize specific antigens, such as tumor-associated antigens (TAAs), for targeted destruction. In cancer vaccines, it is critical to obtain a strong cytotoxic T lymphocyte (CD8+) (Tc) response against the malignant cells. Naive Tc cells are selected and activated by dendritic cells (DC) for their ability to recognize select antigens, and in the case of a vaccine through cross-presentation. The activated Tc cells can then expand, traffic to tumors, recognize tumor cells displaying these antigens, and release chemical factors to induce cancer cell apoptosis. In conventional strategies, soluble TAAs (as recombinant proteins or peptides) are co-administered with DC activators/adjuvants, to improve this process of T cell selection and activation.

As the immune system is designed to respond to pathogenic micro-organisms, there has also been extensive work to adapt pathogenic organisms to elicit anti-tumor immune responses, including both bacteria and viruses, to increase the magnitude of the elicited immune response. Although these cancer vaccines have been shown to elicit CD8 T cell immune responses, the typical response levels generated are usually clinically insufficient to overcome the low immunogenicity and immunosuppressive microenvironment of tumors. Clearly, a major challenge/limitation for conventional cancer vaccines is their potency. One alternative approach for inducing antigen-specific immune responses against cancer cells is the use of nanoparticles, which can be engineered to have specific characteristics, pharmacology, and tropism without the adverse or potential toxicities of pathogenic micro-organism adapted delivery systems.

Enthusiasm for anti-tumor immunotherapy has increased with the clinical success and recent FDA approvals of immune checkpoint inhibitors (e.g., anti-PD-1, anti-CTLA-4) across diverse tumor types. Checkpoint inhibitors release normal immune homeostatic mechanisms on the body's native immune system that impair the anti-tumor immune response, allowing the immune system to augment its preexisting reactivity that can destroy cancer cells. Despite the promise of this approach, as a new paradigm of cancer treatment based on modulating the immune system, the majority of patients do not achieve long-term remission, with only about 20% of patients exhibiting long-term survival at 3 years post-treatment. Furthermore, many cancer types do not respond to this type of therapy, which may be due in part to inadequate education of the immune system to the relevant tumor-associated antigens. Priming the immune system, before and during checkpoint inhibition therapy, to better recognize tumor-associated antigens (TAAs) using cancer vaccines is an attractive option for improving outcomes.

Immune checkpoint inhibitors are not antigen-specific and in fact, their toxicity profile is one of auto-immune reactivity. Indeed, treatments combining multiple checkpoint inhibitors (anti-PD-1 with anti-CTLA-4) show greater efficacy, but also particularly high rates of adverse events and toxicity. Given that both anti-PD-1 and anti-CTLA-4 act by releasing the suppression of general T cell activation and effector functions without priming, the response specifically towards tumors, non-specific immune-related adverse events for this approach are not surprising. This suggests that more powerful checkpoint inhibition is not likely to be safe. Hence, there is a need for additional approaches to anti-tumor immunotherapy.

In one embodiment, the present invention features protein nanoparticles comprising cancer-testis (CT) peptide epitopes within a synthetic nanoparticle. The synthetic nanoparticle has size characteristics that accentuate the uptake and antigen presentation by professional antigen presenting cells such as immune system dendritic cells (DC). The formulation of CT antigens within a nanoparticle can significantly enhance antigen-specific cell-mediated responses (e.g., antigen-specific cytokine response, lysis of specific cancer cells) relative to the conventional CT vaccines. Specifically, a higher degree of lysis of cancer cells bearing the specific antigens was observed using the nanoparticle vaccine, relative to the “conventional” (non-nanoparticle) formulations. Further still, the combination of the two antigens in a vaccine can preserve the increased individual responses that are observed for each antigen alone.

In another embodiment, the present invention utilizes immune checkpoint inhibition combined with strategies to enhance antigen-specificity of the ‘released’ immune response to improve anti-tumor activity. In particular, the invention features a combination of anti-PD-1 treatment with the protein nanoparticle strategy to significantly increase the immune system's ability to respond to TAAs.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide an effective vaccine platform for cancer therapy. In some aspects, the present invention utilizes a nanoparticle to deliver cancer antigens with adjuvants, which can significantly increase specific anti-tumor responses to different cancer antigens. The nanoparticle comprises a protein capsule, to which adjuvants are attached in the internal hollow cavity and cancer epitopes to the surface. For instance, the nanoparticle vaccine platform may include the attachment of cancer-testis (CT) antigens for human-specific CT cancers, which have been demonstrated to be effective target antigens in ex vivo studies.

In an exemplary embodiment, TAAs can be delivered using a protein nanoparticle platform, E2, to elicit antigen-specific recognition and destruction of cancer cells (relative to conventional TAA vaccine formulations without the nanoparticle platform), and significantly extend survival time for tumor-bearing mice in a particularly aggressive tumor model. The present invention has further demonstrated that cytolytic Tc responses, specifically towards cancer cells expressing TAAs, can significantly increase when the TAA peptides are coupled to E2 nanoparticles that are functionalized with a dendritic cell (DC) activator. Improved nanoparticle uptake by DCs appears to induce stronger Tc responses by simultaneously increasing both spatial and temporal dosing of adjuvant activators and antigens to DCs, thereby avoiding the indiscriminate and non-specific immune stimulation of immune adjuvants administered systemically or in the periphery. This approach mimics viruses for which the body is particularly effective at mounting a strong immune response; and since it is not a virus, the possible secondary effects of viral-based vector systems are eliminated. Additional advantages of the protein nanoparticle over other delivery systems include the caged protein design feature limiting systemic exposure of the immune adjuvant (stimulator), tropism for DCs, a high degree of physical stability with controlled release of antigenic peptide, ease of manufacture and quality control, and a particularly flexible and modular platform allowing evaluation of a wide range of modifications.

In other aspects, the present invention also features a novel combination therapy that combines the nanoparticle vaccine platform with checkpoint blockade therapy, which has demonstrated significantly improved results in in vivo murine tumor studies. Without wishing to limit the present invention to a particular theory or mechanism, the cancer vaccine delivery system can be readily combined with immune checkpoint inhibitors without increased toxicity. For instance, the tumor-bearing mice did not show any outward signs of toxicity (e.g. no weight loss, loss of appetite, unusual behavior, etc.) due to treatment with the nanoparticle vaccine. Further still, the immune-activating nanoparticle formulations in combination with anti-PD-1 therapy have demonstrated promise in not only prolonging survival time, but also provide evidence of sustained anti-tumor immune responses and immunologic memory relevant for long-term survival, which can have a significant impact on global health.

The combination of a protein nanoparticle vaccine with the checkpoint inhibitor is counterintuitive because of the potential for undesired effects. Addition of the checkpoint blockade could cause auto-immune reactivity because inhibition of these checkpoints allows any autoreactive cells to become activated and result in autoimmune disease. The combination could even result in a cytokine storm because the extended activation of immune cells can cause a general release of inflammatory cytokines into the bloodstream, causing further inflammation and more cytokine release, referred to as a cytokine storm.

The result of combining the nanoparticle vaccine with immune checkpoint inhibitors is surprising. Without wishing to limit the invention to any theory or mechanism, it is believed that the combined therapy can advantageously increase survival time as well as prevent tumor development in pre-existing tumor conditions, whereas single-therapies only modestly increase survival. For instance, the combination therapy was shown to have a much greater synergistic effect than the individual therapies. As shown in FIG. 7, for nanoparticle vaccine (with gp-100 peptide) alone and PD-1 alone, the survival rates at day 60 were approximately 0% and 10%, respectively. Unexpectedly, the combination group demonstrated 50% survival. This 50% survival at a longer-term timepoint is an unusually high survival rate for the particular aggressive cancer model that was tested, and this high number was surprising.

Furthermore, tumor re-challenge of surviving mice surprisingly shows evidence of T cell memory, which showed complete prevention of tumor development in some animals. For example, mice treated with the combination therapy (anti-PD-1 and nanoparticle with gp-100 antigenic peptide) that had survived the first challenge survived significantly longer than naïve mice (no vaccination), when challenged again with the same dose of tumor cells. This shows particular efficacy in treatment of tumors and prevention of future tumors. This result is surprising because treatment with anti-PD-1 alone is not known to induce a memory response. Although some vaccines are capable of inducing a memory response, E2 has not been previously tested, so the ability of the E2 vaccine to have this effect was unknown. None of the presently known prior references or work has the unique inventive technical feature of the present invention. To their best knowledge, the inventors are not aware of any investigations in which the suppression of checkpoint inhibition therapy (e.g., anti-PD-1) has been synergistically combined with antigen-specific, activation induced by protein nanoparticle vaccines.

The successful use of these human CT antigens in a protein nanoparticle as a vaccine is surprising. It is surprising because human CT antigens are less immunogenic than previously tested non-human SIINFEKL antigen studies. For example, some reasons that peptides from human CT antigens are less immunogenic (than non-human antigens such as SIINFEKL) are that they have low MHC binding affinities, have an intrinsic level of tolerance due to being a self-antigen, and are present in low numbers due to their recognition as a self-antigen. Contrastingly, SIINFEKL is a totally foreign antigen for which there is no intrinsic immune tolerance. Traditionally, it is thought that antigens with peptides which have high affinities and/or come from a foreign source are more immunogenic and thus more suited to development of vaccines. While the highly immunogenic SIINFEKL antigen has been successfully used as a vaccine in a protein nanoparticle, it is surprising that less immunogenic CT antigens can also be used. Contrary to the low-affinity human CT antigens, the OT-1 model with the SIINFEKL peptide selects primarily for T cells recognizing this peptide and has a very high affinity for it. As such, it is surprising that the less immunogenic human CT antigen can be successfully used as vaccine, like the more immunogenic SIINFEKL antigen. The successful result is also surprising because not all CT epitopes that were tested yielded an immunogenic response. For example, the splenocytes from mice immunized with E2 conjugated to peptide epitopes ILTIRLTAA (from NY-ESO-1) and KVAELVHFL (from MAGE-A3) did not yield an antigen-specific IFN-gamma response beyond background.

Additionally, the use of human CT antigens in a protein nanoparticle as a human vaccine is surprising because the field of cancer immunology is a complex and sometimes unpredictable one. Strategies that may appear to work with non-human antigens may not perform well with human antigens. Furthermore, strategies that appear to work in a mouse model may not necessarily perform well in human studies, especially when different antigens are targeted.

In spite of the difference in immunogenicity between SIINFEKL and CT antigens, and the unpredictability of the field, CT antigens were surprisingly found to give excellent results. Specifically, having shown the capacity to elicit antigen-specific immunity to the SIINFEKL peptide, robust immunity was elicited to HLA-A2 binding peptides from the CT antigens NY-ESO-1 and MAGE-A3 in the setting of HLA-A2 transgenic mice over the irrelevant SIINFEKL control peptide. Notably, migration of this strategy and platform to a relevant tumor-associated antigen (gp-100) and B16-F10 melanoma tumor model demonstrated the elicitation of exceptional anti-tumor immunity yielding prolonged survival and evidence of immunologic immunity in this highly aggressive tumor model

This survival data was achieved with use of an antigen derived from murine gp-100, a melanoma-associated antigen that is not necessarily a CT antigen per se. The studies with human CT antigens were conducted in HLA-A2 transgenic animals so that the murine T cell repertoire developed with the capacity to recognize antigenic peptides in the context of HLA-A2 (positive selection).

### BRIEF DESCRIPTION OF THE DRAWINGS

The features and advantages of the present invention will become apparent from a consideration of the following detailed description presented in connection with the accompanying drawings in which:

FIG. 1 is a non-limiting schematic (with TEM image) of E2 protein nanoparticles designed for eliciting dendritic cell (DC) activation and cross-presentation, resulting in generation of antigen-specific CD8+ T cell responses. E2 is used to simultaneously deliver tumor-associated antigens (peptide, externally) and adjuvant (CpG, internally). Antigen-specific immune responses to these nanoparticles were significantly higher than those induced by antigen and adjuvant formulations that were not conjugated to E2.

FIG. 2 shows a non-limiting embodiment of a combination therapy in accordance with the present invention.

FIG. 3A shows vaccine components per dose of different formulation groups (a-f).

FIG. 3B is a summary of averaged ELISpot data, which evaluated antigen-specific IFN-γ secretion. HLA-A2 mice were immunized with different formulations (a-f), and splenocytes were pulsed ex vivo in the presence of relevant peptide (NYESO) or irrelevant peptide (SIINFEKL) and analyzed for specific IFN-γ secretion. Higher NY-ESO-1 epitope-specific IFN-γ secretion was observed for the group that received CpG-NYESO-E2. Data is presented as average±S.E.M. of at least 3 independent experiments (n>3). Statistical significance was determined by two-way ANOVA followed by a Tukey's test (***p<0.001; ****p<0.0001).

FIG. 4A shows vaccine components per dose of various formulation groups (a-e).

FIG. 4B is a summary of averaged ELISpot data, which evaluated antigen-specific IFN-γ secretion. HLA-A2 mice were immunized with different formulations (a-e), and splenocytes were pulsed ex vivo in the presence of relevant peptide (MAGE) or irrelevant peptide (SIINFEKL) and analyzed for specific IFN-γ secretion. Higher MAGE-A3 epitope-specific IFN-γ secretion was observed for the group that received CpG-MAGE-E2. Data is presented as average±S.E.M. of at least 3 independent experiments. Statistical significance was determined by two-way ANOVA followed by a Tukey's test (**p<0.01; ***p<0.001).

FIG. 5A shows vaccine components per dose of different formulation groups (a-f).

FIG. 5B is a summary of averaged ELISpot data: which evaluated antigen-specific IFN-γ secretion. HLA-A2 mice were immunized with different formulations (a-f). Splenocytes of immunized mice were pulsed ex vivo in the presence of relevant peptide (NYESO or MAGE) or irrelevant peptide (SIINFEKL) and analyzed for specific IFN-γ secretion. Data is presented as average±S.E.M. of at least 3 independent experiments. Statistical significance was determined by two-way ANOVA followed by a Bonferroni's test (**p<0.01; ***p<0.001).

FIG. 6 shows an experiment schedule for mice that were inoculated with 10⁴ B16-F10 and assigned to treatment groups of PBS, CpG-gp-E2, Anti-PD1, or combination of CpG-gp-E2 and anti-PD1.

FIG. 7 shows treatment results for the vaccine+checkpoint blockade inhibitor combination therapy−consolidated data of 2 independent experiments, showing 50% remission of tumors in a particularly aggressive tumor model. Data is for a B16-F10 tumor model in C57BL/6 mice, with N=10. Mice were treated with nanoparticle vaccine alone (CpG-gp100-E2), anti-PD1 alone, combined therapy (CpG-gp100-E2 with anti-PD1), and PBS control. C57BL/6 mice were subcutaneously (SC) inoculated with 10⁴ B16-F10 cells (Day −1). On Day 0, CpG-gp100-E2 and combined groups were immunized with 50 μg CpG-gp100-E2, followed by a booster seven days after the prime immunization. For the anti-PD1 and combined groups, 100 μg of anti-PD-1 antibody was injected (IP) two days after the vaccine booster and repeated every three days×5. Animals and tumor sizes were examined for 60 days, with animals being sacrificed when the tumor reached size limit. Statistical significance was determined by the log-rank test, with p<0.05 considered significant.

FIG. 8 data from a re-challenge study of mice that survived the combination study were re-inoculated on day 64 (considered as Day 0 on the re-challenge study) with 10⁴ B16-F10, with no additional anti-cancer treatments. Results show immunological memory was obtained from the original round of therapy.

### DESCRIPTION OF PREFERRED EMBODIMENTS

Disclosed are the various compounds, solvents, solutions, carriers, and/or components to be used to prepare the compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering” and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, intranasally, topically, or the like.

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be cancer, such as melanoma, colon, bladder, lung, prostate, or breast cancers. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for cancer can be identified by, for example, any or a combination of appropriate diagnostic or prognostic assays known in the art.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The compositions described herein can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in d deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical compositions can include additional carriers, as well as thickeners, diluents, buffers, preservatives, surface active agents and the like in addition to the compounds disclosed herein. Pharmaceutical compositions can also include one or more additional active ingredients such as antimicrobial agents, anti-inflammatory agents, anesthetics, and the like. The pharmaceutical compositions can be administered in a number of ways and dosages depending on whether local or systemic treatment is desired, and the area/location to be treated.

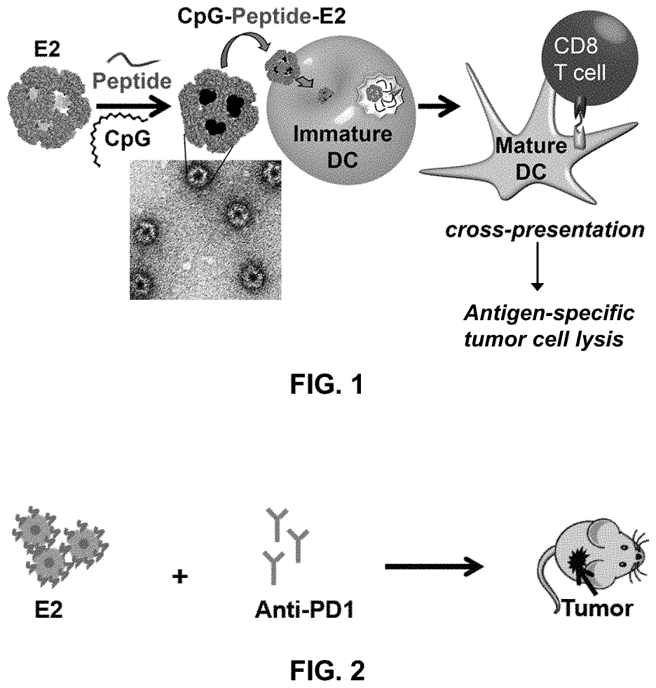

Referring now to FIG. 1, in some embodiments, the present invention features an immunotherapy composition comprising a non-viral nanoparticle vaccine comprising one or more cancer-testis (CT) antigens bound to a nanoparticle. In other embodiments, the present invention may also feature a method of treating cancer in a subject in need thereof. The method may comprise administering to the subject a therapeutically effective amount of the immunotherapy composition comprising the non-viral nanoparticle vaccine to elicit antigen-specific recognition.

Referring to FIG. 2, in some embodiments, the present invention may feature an immunotherapy composition for treating cancer, comprising a non-viral nanoparticle vaccine and one or more immune checkpoint inhibitors. The non-viral nanoparticle vaccine may comprise one or more antigens bound to a nanoparticle. According to other embodiments, the invention includes a method of treating cancer in a subject in need thereof, comprising administering to the subject a therapeutically effective amount of an immunotherapy composition comprising a non-viral nanoparticle vaccine and one or more immune checkpoint inhibitors. The nanoparticle vaccine may be administered separately or in conjunction with the immune checkpoint inhibitors. Without wishing to limit the invention, administration of the immunotherapy composition can elicit antigen-specific recognition while simultaneously blocking checkpoints to remove immune suppression.

In some embodiments, the nanoparticle may be an E2 subunit of a pyruvate dehydrogenase complex. The nanoparticle may comprise a dodecahedral caged structure with a diameter of about 25 nm, and an internal 12-nm cavity with about 5-nm openings leading to said cavity. In some embodiments, the antigens may be bound to an external surface of the nanoparticle.

In some embodiments, the one or more antigens may be tumor-associated antigens (TAAs). Examples of TAAs include, but are not limited to, any solid tumor-associated antigen containing an HLA Class I binding epitope, gp70 epitopes, gp100 epitopes, NY-ESO-1 epitopes, MAGE-A3 epitopes, oncofetal proteins such as carcinoembryonic antigen (CEA), overexpressed antigens such as Survivin, cancer-testis antigens, and lineage-restricted antigens such as prostate-specific antigen (PSA). For example, the TAAs may comprise cancer-testis antigens such as epitopes of MAGE-A(1, 3, or 4), MAGE-C1, NY-ESO-1, CT-7, CT-10, CT-83, SSX-1, SSX-2, SSX-4, SCP-1, PRAME, or a combination thereof. In other embodiments, the one or more checkpoint inhibitors may be inhibitors of PD-1 (CD279), BTLA (CD 272), CTLA-4 (CD152), TIM-3, TIGIT, LAG-3 (CD223), CD96, CD200R, MOG/BtnI2 (CD277) CD112, CD155 and their respective receptors (characterized and non-characterized), or ligands for receptors for PD-L1, PD-L2, B7-X, B7-H3, B7-H5, B7-H5, B7-H7, or adenosine.

In some other embodiments, the nanoparticle vaccine may further comprise an adjuvant disposed within the nanoparticle. A non-limiting example of the adjuvant is CpG. In some embodiments, the antigens and adjuvants may be conjugated to the nanoparticle via linkers that are responsive to an acidic and reducing environment, respectively, of an endosome after dendritic cell uptake.

Protein nanoparticulate systems have benefits that are desired in a vaccine delivery vehicle. Protein based size distribution, and can be modified through protein engineering to enable precise site-specific functionalization. Particles ranging from about 20-200 nm can accumulate faster and at higher concentrations in the lymph nodes (areas of high DC activity), and that particles between 20-45 nm are taken up by 50% of lymph node-residing DC (vs. only 5% for 100 nm particles). The increased uptake yields significantly higher humoral and cellular immunity, thereby eliciting responses that are higher than those obtained by commonly-used adjuvants. Therefore, protein nanoparticles, which are often within this desired size range of 20-45 nm, are ideally sized for DC delivery. Additionally, protein nanoparticles can also be functionalized in both the internal cavity and the outer surface. This gives extra capacity for coupling exogenous activation molecules, potentially at higher amounts than in solid nanoparticle formulations. The biocompatibility from biodegradation products is high with respect to cytotoxicity because they are comprised of amino acids.

One nanoparticle with favorable DC uptake properties is the E2 nanoparticle. The E2 nanoparticle is a non-viral, effective delivery system for delivering antigens for immunotherapy applications. This self-assembled protein nanoparticle is derived from the E2 subunit of the pyruvate dehydrogenase complex from the thermophile Bacillus stearothermophilus. The gene for E2 has been truncated to only the structural core and the protein has been expressed in E. coli. The assembled nanoparticle is comprised of 60 identical monomers that form a dodecahedral caged structure that is very stable over the pH 3 to 8 and temperatures up to 80° C. Although mammals also utilize pyruvate dehydrogenase, this protein from B. stearothermophilus has only 32% sequence homology with the human form. E2's caged structure has a diameter of about 25 nm, which is within the favored size range for lymphatic transport and DC uptake, and an internal 12-nm cavity with about 5-nm openings leading to this hollow cavity. It has three interfaces (internal hollow cavity, subunit-subunit interface, exterior surface) that can be modified to impart new functional elements, such as pH-dependent drug release, triggered particle disassembly, and cell targeting. This structure can be manipulated extensively for drug delivery functionality.

By co-delivering antigens within the E2 non-viral protein nanoparticle designed to emulate the spatial and temporal delivery features of a virus, the cell-mediated, anti-tumor responses were significantly enhanced. Without the E2 nanoparticle, the free antigen and free adjuvant gave activation levels close to negative controls (buffer-only). Referring to FIG. 1, TAAs (e.g., gp100, NY-ESO-1, MAGE-A3 epitopes) may be attached to the external surface and an activating adjuvant (e.g., CpG) to the interior of the E2 nanoparticle. The antigens and adjuvants are conjugated through linkers that are responsive to the acidic and reducing environment, respectively, of the endosome after DC uptake.

Cancer-Testis (CT) Antigen Vaccines

In some embodiments, the present invention features compositions of CT antigens conjugated to a nanoparticle. In other embodiments, the composition may further include an adjuvant. In an exemplary embodiment, human-specific TAAs, and in particular, for the human cancer-testis (CT) class of antigens (NY-ESO-1 and MAGE-A3) and DC-activating DNA (CpG; ODN 1826) were both conjugated to E2, which resulted in a significantly higher DC activation and antigen cross-presentation. CT antigens are good targets because normal adult tissue expression of CT antigens is limited only to testes, which is an immune privileged site. While other tumor-associated antigens (TAAs) are often expressed at low levels in healthy tissue, expression of CTs is restricted only to cancer cells and the immune-privileged cells in the testis. Furthermore, CT antigens exist in a high proportion of different human tumors such as melanoma, bladder, lung, prostate, and breast cancers. In particular, NY-ESO-1 is expressed in 82% of neuroblastomas and 46% melanomas while MAGE-A3 is also expressed in 76% of melanoma cancers.

To the knowledge of the inventors, the present invention is the first of its kind to feature clinically-tested human cancer-testis (CT) peptide epitopes within a synthetic nanoparticle. In one embodiment, two significant clinical CT targets, the HLA-A2 restricted epitopes of NY-ESO-1 and MAGE-A3 were used in the viral-mimetic packaging strategy. A transgenic mouse model humanized with the HLA-A2 gene had significantly higher IFN-γ secretion and cell lysis activity by splenocytes when the immunodominant epitopes of CT antigens and CpG were coupled to E2, relative to controls. Furthermore, the combined delivery of epitopes from different antigen sources within E2 yielded an additive lytic effect towards human cancer cells bearing those antigens. As such, the present invention has shown that the formulation of CT antigens within a nanoparticle can significantly enhance antigen-specific cell-mediated responses (e.g., antigen-specific cytokine response, lysis of specific cancer cells), and the combination of the two antigens in a vaccine can preserve the increased individual responses that are observed for each antigen alone. Specifically, there was a higher degree of lysis of cancer cells bearing the specific antigens using the nanoparticle vaccine, relative to the “conventional” (non-nanoparticle) formulations.

The E2-based vaccines have clearly demonstrated an ability to strongly activate specific anti-TAA responses, showing that E2-formulated vaccines alone are potent enough to significantly extend survival time of animals in an aggressive cancer model. By combining immune checkpoint inhibitors with the vaccine's ability to selectively activate against a specific TAA, anti-tumor activity can be further improved. The following section examines combination therapy using epitopes from the TAA gp100 in the B16-F10 tumor model and from gp70 in the CT26 tumor model together with anti-PD-1.

Combination Therapy:

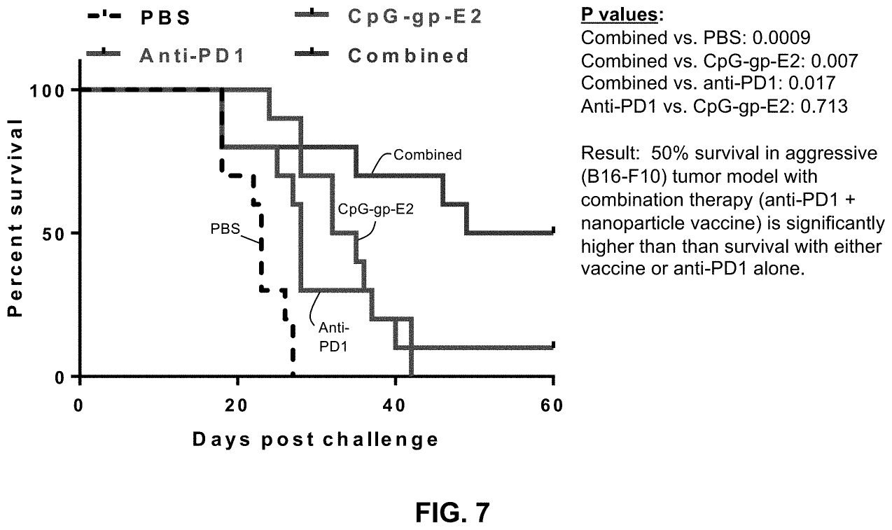

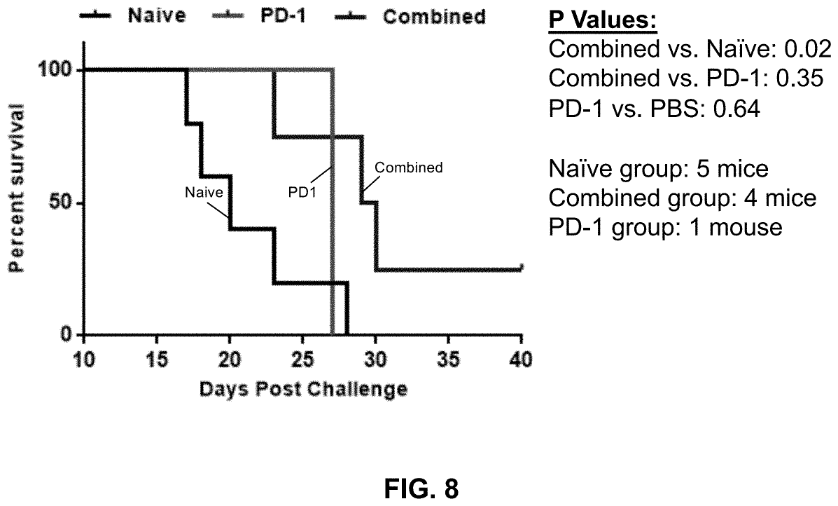

In some embodiments, the present invention features a therapeutic combination of E2-based vaccine formulations with a checkpoint inhibitor, such as anti-PD-1, for enhanced anti-tumor immune response and activity and long-term survival outcomes. The E2-based vaccines have clearly demonstrated an ability to activate specific anti-TAA responses, showing that E2-formulated vaccines alone are potent enough to significantly extend survival time of animals in the B16-F10 cancer model, relative to non-E2 vaccines. Current strategies using checkpoint blockades (e.g., anti-PD1, anti-CTLA4), while effective for ˜20% of patients, still do not result in long-term remission for the majority of patients. Furthermore, for the aggressive B16-F10 tumor model, it is uncommon to obtain a “cure” for tumors. Referring to FIGS. 7-8, in contrast, the data shows no observable tumor growth for ˜50% of the mice treated with combination therapy, which supports that combination therapy of the E2 antigen-delivery particle with anti-PD1 can treat pre-existing tumor in mice to give an improved outcome than treatment with anti-PD1 alone. In addition, subsequent tumor re-challenge studies suggest that T-cell memory can also be elicited in this approach to protect the mice upon tumor re-challenge.

Immune checkpoint inhibitors are not antigen-specific and in fact, their toxicity profile is one of auto-immune reactivity. Indeed, treatments combining multiple checkpoint inhibitors (anti-PD-1 with anti-CTLA-4) show greater efficacy, but also particularly high rates of adverse events and toxicity. Given that both anti-PD-1 and anti-CTLA-4 act by releasing the suppression of general T cell activation and effector functions without priming the response specifically towards tumors, non-specific immune-related adverse events for this approach are not surprising. This suggests that more powerful checkpoint inhibition is not likely to be safe. Thus, it is desirable to have immune checkpoint inhibition combined with strategies to enhance antigen-specificity of the ‘released’ immune response, which could have improved anti-tumor activity for more cancer patients. Without wishing to limit the present invention, it is believed that the combination of anti-PD-1 treatment with the E2 nanoparticle vaccines can significantly increase the immune system's ability to respond to tumor-associated antigens while limiting the severe non-specific side effects. Consistent with this, no obvious side effects or adverse reactions in vivo were observed in the murine studies of the invention.

In some embodiments, the combination therapy of the present invention may utilize different antigen targets (beyond gp100), different formulations, different adjuvants, etc., to optimize vaccine response. In other embodiments, the combination therapy may include different checkpoint inhibitors.

### EXAMPLES

The following are non-limiting examples of the nanoparticle vaccine and combination therapy of the present invention. It is understood the examples are not intended to limit the invention in any way. Equivalents or substitutes are within the scope of the invention.

### Example 1: CT Antigen Nanoparticle Vaccines

The target epitopes in this current study are HLA-A2 restricted peptide sequences from New York esophageal squamous cell carcinoma-1 (NY-ESO-1) and melanoma antigen family A, 3 (MAGE-A3). NY-ESO-1 is expressed in 82% of neuroblastomas and 46% melanomas while MAGE-A3 is also expressed in 76% of melanoma cancers. A phase II clinical trial of NY-ESO-1/ISCOMATRIX vaccine which was recently completed in June 2017 (NCT00518206) resulted in 4% partial response (based on a standard of 30% reduction in tumor size), 48% stable disease, and 48% progressive disease; this result highlights the generation of response to NY-ESO-1, but also the need and potential for development of alternative strategies that will yield more effective therapies. Given the wide range of tumors that express CT antigens, their relatively high level in cancer, their restricted expression, and their potential for vaccine improvement, the CT class of antigens is an important and significant clinical target. This example examines the feasibility of using the E2 nanoparticle to induce cell-mediated immune responses against NY-ESO-1 and MAGE-A3 in a mouse model that is transgenic for the human major histocompatibility complex, HLA-A2.

This example also investigates the extent of cell-mediated and cytolytic responses by simultaneous delivery of NY-ESO-1- and MAGE-A3-containing nanoparticles. Tumor escape after single-epitope vaccination is common since cancers often lose expression of the targeted antigen to evade the immune system. Immunization with combined antigens can possibly decrease the risk of tumor escape resulting from antigen loss. Furthermore, increasing the number of different antigen targets in a vaccine can induce a broader range of T cell responses simultaneously, which could be effective in a higher number of patients. Because there is a lack of immune-competent murine tumor models expressing these CT antigens to examine in vivo anti-tumor efficacy in the most physiologically relevant way possible, lytic ability ex vivo was examined using human cancer cell lines expressing both NY-ESO-1 and MAGE-A3.

### Experimental

Materials

Reagents were purchased from Fisher Scientific unless otherwise noted. Complete RPMI used in this study for splenocytes was compromised of RPMI 1640 (Mediatech) with 10% heat-inactivated FBS (Hyclone), 1 mM sodium pyruvate (Hyclone), 100 mg/ml of streptomycin (Hyclone), 0.1 mM nonessential amino acids (Lonza), 2 mML-glutamine (Lonza), and 100 units/ml penicillin. Cancer cell lines used in this study were cultured in DMEM media (Sigma) supplemented with 10% heat-inactivated FBS (Hyclone).

Peptides and CpG

CpG 1826, a bacterial DNA ligand for TLR9, was purchased from Invivogen, and 50 benzaldehyde-modified CpG 1826 with a phosphorothioated backbone was synthesized by Trilink. The NY-ESO-1 and MAGE-A3 peptide epitopes were synthesized by Genscript or Genemed Synthesis (Table 1). Peptides were synthesized both with and without an N-terminal cysteine; the thiol on the cysteine modified peptides was used for conjugation to E2, whereas peptides with no cysteine were used as controls. The abbreviation (e.g., NYESO, MAGE) refers to the peptide, while the names NY-ESO-1 and MAGE-A3 refer to the whole protein.

TABLE 1
List of peptide epitopes sequences and their
respective abbreviations in this study.
Conventional single-letter abbreviations for
amino acids (aa) are used in the peptide
epitope sequence.

| Abbreviation | Antigen source | Sequence | Serotype |
|---|---|---|---|
| NYESO | NY-ESO-1 | C-SLLMWITQV | HLA-A2 |
| | | (aa 157-165) | |
| NYESO (p2) | NY-ESO-1 | C-ILTIRLTAA | HLA-A2 |
| | | (aa 132-140) | |
| MAGE | MAGE-A3 | C-FLWGPRALV | HLA-A2 |
| | | (aa 271-279) | |
| MAGE (p2) | MAGE-A3 | C-KVAELVHFL | HLA-A2 |
| | | (aa 112-120) | |

E2 Purification and Characterization

The D381C mutant of the E2 nanoparticle was used, which has an aspartic acid-to-cysteine mutation at position 381 in the internal hollow cavity of the nanoparticle. The cysteine of D381C can be used for site-directed conjugation, and this nanoparticle is abbreviated as “E2” herein. Expression, purification, and characterization of E2 (D381C mutant) were performed as follows: E. coli strain BL21(DE3) containing the E2 gene was cultured in Luria-Bertani medium containing 100 mg/ml ampicillin. Expression was induced by adding 1 mM of IPTG when the culture reached the optical density of 0.6-0.9 measured at 600 nm. Cells were harvested and stored at 80° C. Cells were lysed using a French pressure cell (Thermo Scientific), and the insoluble fraction was removed by centrifugation. The soluble fraction was heated at 70° C. for 20 min. Denatured native E. coli protein aggregates were removed by centrifugation. The recovered supernatant was loaded to a HiPrep Q Sepharose anion exchange column followed by a Superose 6 size exclusion column. Purity and the molecular weight of purified E2 were confirmed with SDS-PAGE and electrospray ionization mass spectrometry. Dynamic light scattering and transmission electron microscopy were used to check the size, assembly, and monodispersity of the particles. Lipopolysaccharide was removed using Triton X-114 (Sigma) extraction, and endotoxin levels were evaluated using an LAL ToxinSensor kit (Genscript).

CpG and Peptides Conjugation

CpG 1826 modified with a 50-benzaldehyde was attached to the TCEP-reduced cysteines in the internal cavity of E2 nanoparticles using a N-b-maleimidopropionic acid hydrazide (BMPH) linker. The average number of CpG molecules conjugated to the internal cavity of E2 nanoparticle was estimated with intensity analysis in ImageJ software, using standardized concentrations. Peptides with N-terminal cysteines were conjugated to the native lysines on the surface of the E2 nanoparticle by mixing the nanoparticle with a sulfo-SMCC linker in the presence of a 10-fold excess of TCEPM reduced peptides (relative to E2 monomer), and incubating overnight at 4° C. HPLC was used for peptide quantification.

Mice

Transgenic mice expressing the human HLA-A2 gene were obtained from Jackson Laboratory. All animal studies were carried out in accordance with protocols approved by the Institute for Animal Care and Use Committee (IACUC) at the University of California, Irvine. Briefly, 6-8 week old female-HLA-A2 transgenic C57BL/6 mice were immunized subcutaneously at the base of the tail at Day 0. A priming immunization was followed by a booster after 14 days. Injections were 120 ml and contained specific amounts of peptide, E2, and CpG, based on the formulations tested. Seven days after the last immunization, mice, were sacrificed and spleens were isolated.

IFN-γ ELISpot

For IFN-γ ELISpot, a Ready-Set-Go kit (eBioscience) was used. Single-cell suspensions in RPMI were prepared from the spleens isolated from immunized mice, and added at 5×10⁵ and 10⁶ cells/well to PVDF ELISpot plates that were pre-coated with an antimouse IFN-γ antibody. Cells were incubated with either 10 mg/ml of the relevant peptide or an irrelevant peptide (SIINFEKL) for 24 h at 37° C. Unstimulated cells in RPMI were plated and served as a negative control. Positive control wells contained 2% PHA-M (Gibco). IFN-γ spots were developed following the manufacturer's protocol. Plates were scanned and quantified using an ELISpot reader (Cellular Technology) and immunospot analysis software (Immunospot Analysis Pack).

Cell Lines

A375, a human malignant melanoma cell line, and MCF-7, a human breast cancer cell line, were purchased from ATCC. A375 was cultured in DMEM supplemented with 10% FBS, and MCF-7 was cultured in DMEM supplemented with 10% FBS and 0.01 mg/ml human recombinant insulin. Cells were incubated at 37° C., under 5% CO₂, and were passaged 2-3 times a week.

Cell Lysis Assay

Single cell suspensions prepared from splenocytes isolated from immunized mice were cultured in RPMI at 5×10⁶ cell/ml and incubated overnight at 37° C. On day 1, 10 mg/ml of target peptide was added to the cells, incubated for 24 h at 37° C., and washed twice with PBS to remove the unbound peptide. On day 3, the culture was supplemented with 0.4 ng/ml of IL-2. Peptide stimulated splenocytes were collected on day 5 to perform the cytotoxicity assay. LDH release was measured with a colorimetric assay, CytoTox 96 (Promega), to examine the specific lysis of target cancer cell lines expressing NY-ESO-1 and MAGE-A3 antigens. A375 expresses both NY-ESO-1 and MAGE-A3, while MCF7 has low expression of these antigens. Splenocytes were counted with a hemocytometer, co-cultured with the target cancer cell lines at effector-to-target ratios of 100:1, 50:1, and 25:1, and evaluated for LDH release following the manufacturer's protocol.

Statistical Analysis

Statistical analysis was carried out using GraphPad Prism. Data are presented as mean±standard error of the mean (S.E.M.) of at least three independent experiments (n>3). Statistical analysis was determined by a two-way ANOVA over all groups followed by a Tukey's multiple comparison test, unless otherwise noted. P-values less than 0.05 were considered significant.

Results

Conjugation of CpG and Cancer-Testis Peptides to E2 Nanoparticle Yielded Intact Nanoparticles

Both CpG and peptides were successfully conjugated to the E2 nanoparticles. The number of peptides conjugated to the E2 nanoparticle was quantified with HPLC, and found that on average, 140±16 NYESO or 155±21 MAGE peptides were attached on each protein nanoparticle. Conjugation of CpG and peptides to the E2 nanoparticle resulted in a 1:1 mass ratio. Dynamic light scattering revealed a hydrodynamic diameter of 28.4±0.7, 30±1.3, and 30±0.9 nm for E2, CpG-NYESO-E2, and CpG-MAGE-E2 respectively. DLS data confirmed that particles remained unaggregated and within the optimal reported size for lymphatic drainage (20-45 nm), even after conjugation. This verifies that attachment of short peptides (9 amino acid length) on the surface of the nanoparticle does not result in a dramatic change in size. TEM analysis further confirmed intact, nonaggregated CpG-NYESO-E2 and CpG-MAGE-E2 nanoparticles. The zeta potential of E2, CpG-NYESO-E2, and CpG-MAGE-E2 nanoparticles were −11.7±1 mV, −12.8±1 mV, and −11.1±1.8 mV, respectively. This data confirmed that conjugation of NYESO or MAGE peptides on the surface and CpG inside of the nanoparticles did not change the overall surface charge as compared to the E2 nanoparticle itself.

Immunization with CpG-NYESO-E2 Nanoparticles Yielded Increased Antigen-Specific IFN-γ Secretion

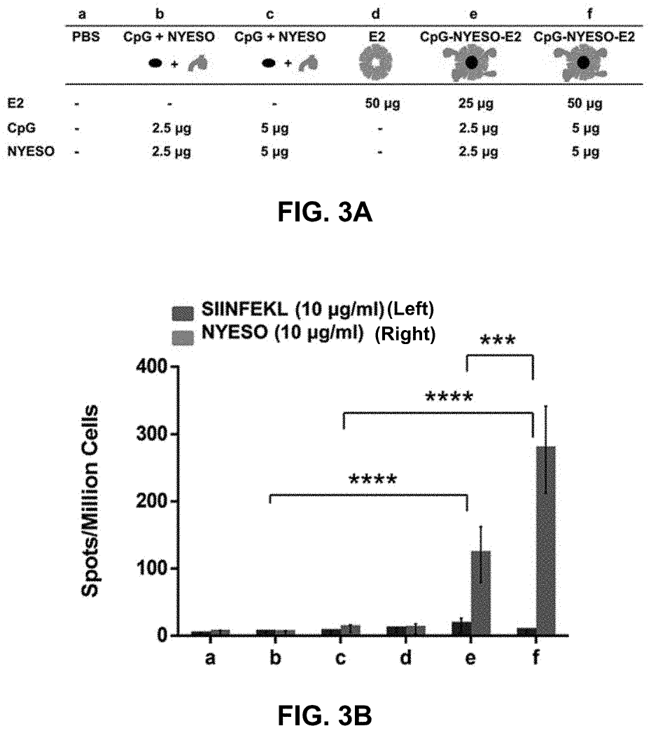

Referring to FIG. 3A, different vaccine formulations of NY-ESO-1 antigen peptide, CpG, and E2 were investigated. As shown in FIG. 3B, immunization with the CpG-NYESO-E2 nanoparticle significantly increased the NY-ESO-1 epitope-specific IFN-γ secretion by 25-fold, compared to immunization with unbound CpG and NYESO, at an equivalent amount of antigen and adjuvant. In contrast, negligible IFN-γ response by the cells pulsed with an irrelevant SIINFEKL peptide was observed, confirming that the response generated from immunization was specific to the NYESO epitope. Splenocytes isolated from mice immunized with bare E2 nanoparticle also lacked significant amounts of NYESO-specific IFN-γ secretion, confirming that higher IFN-γ secretion resulting from CpG-NYESO-E2 immunization was not a result of non-specific immune responses to the E2 delivery platform itself. This observed increase in IFN-γ secretion may result from higher DCs activation and more efficient cross-presentation of the NYESO epitope.

Higher peptide-specific IFN-γ secretion for the group receiving immunizations of 50 mg CpG-NYESO-E2 was observed, as compared to 25 mg CpG-NYESO-E2 (FIG. 3B, groups e, f). This demonstrates dose dependency of the generated cell-mediated immune response to the NYESO epitope. However, immunization with 100 mg of CpG-NYESO-E2 did not increase the IFN-γ secretion compared to 50 mg, and in fact showed a significant decrease across the cohort of mice. This could be due to increased levels of suppressive T cells, T cell exhaustion, or high antigen doses leading to increase tolerance.

Higher Lysis Activity Toward NY-ESO-1 Cancer Cells was Observed for the Group Immunized with CpG-NYESO-E2 Nanoparticles

Lytic capacity of splenocytes isolated from immunized mice was tested on a human melanoma cell line expressing NY-ESO-1 (A375) and on a human breast cancer cell line negative for NY-ESO-1 expression (MCF-7), both positive for HLA-A2. The increase in antigen-specific IFN-γ levels that resulted from CpG-NYESO-E2 immunization translated to a 15-fold increase in the lysis activity towards A375, compared to unbound peptide and CpG. In contrast, no significant lysis was observed for the control cell line, MCF-7. This supports the specificity of the immune response for the NY-ESO-1 antigen. Splenocytes isolated from mice immunized with bare E2 nanoparticles or antigen/CpG alone did not increase specific lysis; this confirms that the significant increase in lysis resulting from the CpG-NYESO-E2 immunization is due to the complete nanoparticle-adjuvant-antigen delivery system, and not the effect of E2 or antigen alone. Taken together, the present invention shows that conjugation of the NY-ESO-1 epitope and CpG adjuvant to the nanoparticle is an effective delivery strategy for increasing INF-γ response, and it results in a functional lysis activity that is specific towards the NYESO-1 epitope.

Immunization with CpG-MAGE-E2 Nanoparticles Yielded Increased Antigen Specific IFN-γ Secretion

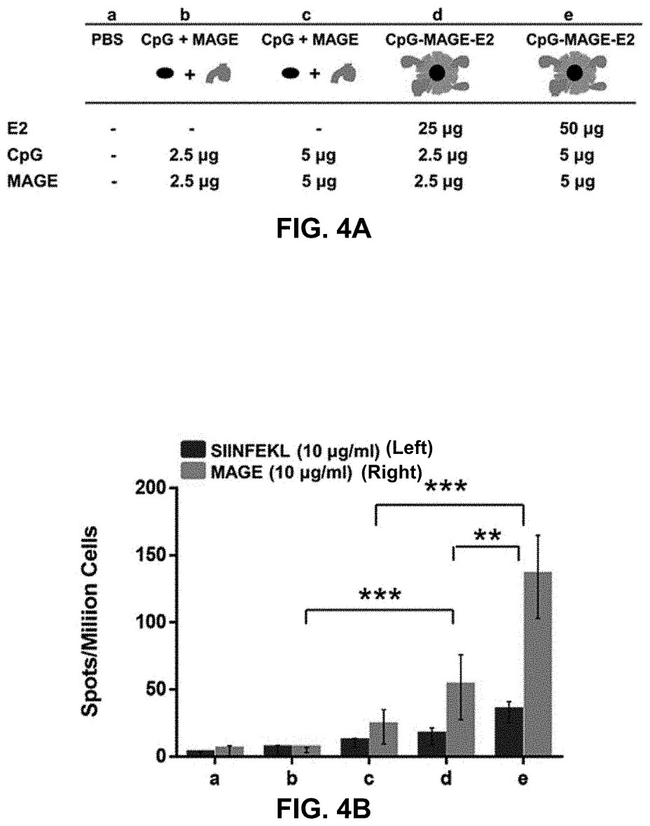

Referring to FIG. 4A, peptide epitopes for MAGE-A3 were conjugated to the E2 nanoparticle bearing the CpG adjuvant. The ELISpot results of immunizations with the different MAGE formulations are presented in FIG. 4B. Vaccination with the CpG-MAGE-E2 nanoparticles increased the number of IFN-γ spots, relative to free CpG and MAGE. A dose-dependent response was observed where significantly higher IFN-γ secretion was obtained for mice immunized with 50 mg compared to the 25 mg dose. Statistical analyses show that IFN-γ levels resulting from the irrelevant SIINFEKL control peptide for mice immunized with 50 mg CpG-MAGE-E2 (group e) is not significantly different compared to the other vaccine formulations in FIG. 4B. Similar to CpG-NYESO-E2, CpG-MAGE-E2 increased the specific IFN-γ secretion compared to the free peptide/CpG control group. A high IFN-γ secretion to the MAGE epitope was obtained without a need for T helper peptides or a separate DNA vaccine.

Higher Lysis Activity Toward MAGE-A3 [symbol] Cancer Cells was Observed for the Group Immunized with CpG-MAGE-E2 Nanoparticles

Splenocytes isolated from mice immunized with CpG-MAGE-E2 nanoparticles significantly enhanced the lytic activity toward A375 by 9-fold at a 100:1 effector-to-target ratio, compared to free peptide and CpG. As expected, lytic activity observed for the mice immunized with CpG-MAGE-E2 is specific to the cell line expressing MAGE, where no significant lysis was observed for the control cell line MCF-7. As with the results for the NY-ESO-1 antigen on the nanoparticle, the lysis activity toward target cell line is dose dependent. Data for the MACE epitope, together with the data generated for the NYESO epitope, confirm that the E2 nanoparticle can be used as an effective platform to simultaneously deliver clinically-tested CT antigens and CpG, resulting in an increase in the cellular-mediated immune responses generated to the target epitope.

Co-Immunization with Nanoparticles Bearing Both NYESO and MAGE Epitopes Yielded an Additive IFN-γ Effect and Increased the Lysis Activity

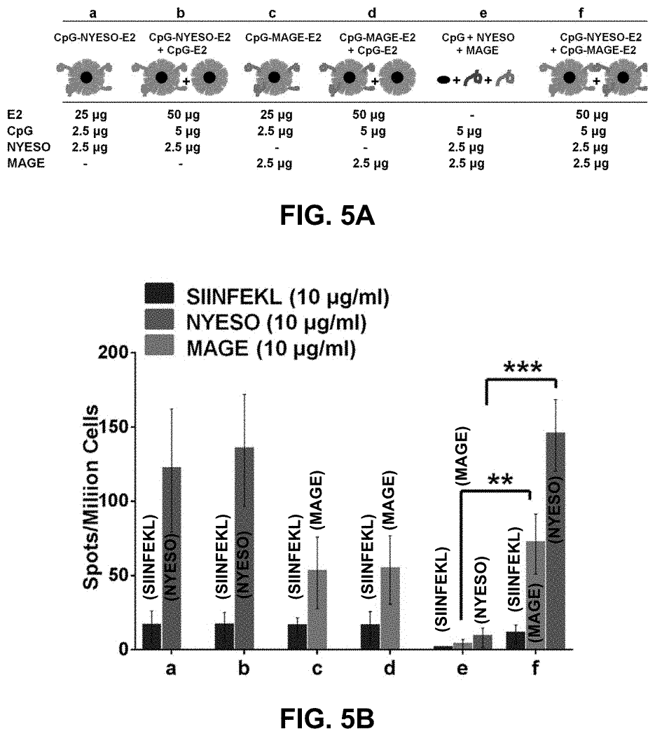

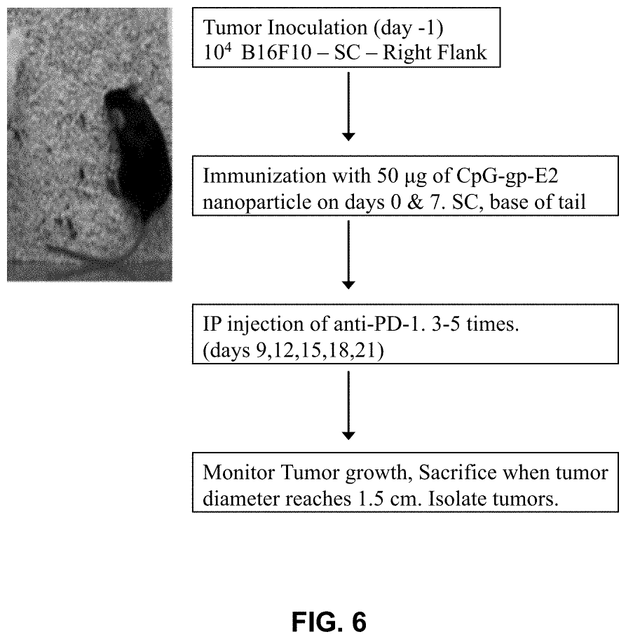

Referring to FIG. 5A, the effects of simultaneous immunization with CpG-NYESO-E2 and CpG-MAGE-E2 nanoparticles was investigated. It was observed that immunization with the CpG-NYESO-E2 and CpG-MAGE-E2 nanoparticles together (25 mg each, 50 mg total per dose) significantly increased the NYESO and MAGE epitope-specific IFN-γ secretion, compared to immunization with simultaneous vaccination of unbound CpG, NYESO, and MAGE peptides, as shown in FIG. 5B. Negligible IFN-γ for the cells pulsed with an irrelevant SIINFEKL peptide was observed, confirming the specificity of immune response generated from immunization. The same levels of IFN-γ secretion to the individual NYESO and MAGE epitopes were obtained when CpG-NYESO-E2 and CpG-MAGE-E2 nanoparticles were co-administered, relative to each nanoparticle formulation separately; this shows that the specific IFN-γ responses to individual epitopes were preserved after co-immunization. The result is an additive effect to IFN-γ secretion, with the total IFN-γ frequencies of group f (sum of NYESO- and MAGE-specific IFN-γ) being approximately equal to the sum of individual NYESO-specific IFN-γ (group b) and MAGE-specific IFN-γ (group d). Furthermore, this additive effect to IFN-γ secretion from the simultaneous immunization is entirely antigen specific and is not due to any adjuvant effect from the CpG-E2 nanoparticle itself, since the addition of CpG-E2 alone did not promote further specific IFN-γ secretion (compare formulations a to b, and c to d in FIG. 5B).

Consistent with the ELISpot data, splenocytes isolated from mice simultaneously immunized with CpG-NYESO-E2 and CpG-MAGE-E2 nanoparticles (25 mg each, 50 mg total per dose) significantly enhanced the lytic activity toward A375, in a dose dependent manner, relative to unbound CpG, NYESO, and MAGE epitopes. The lytic activity was specific to A375, with no lysis observed for the control cell line. Furthermore, an elevated and additive lysis activity was observed for the mice that were co-immunized with CpG-NYESO-E2 and CpG-MAGE-E2 formulations compared to each formulation separately. However, co-immunization with higher doses of CpG-NYESO-E2 and CpG-MAGE-E2 nanoparticles (50 mg each, 100 mg total per dose) did not amplify the specific-IFN-γ secretion or lysis activity, relative to each formulation separately; in fact, both IFN-γ and lysis effects were lower than the effects of each individual antigen-nanoparticle alone. This data, together with the data for the 100 mg CpG-NYESO-E2, suggests that for the current immunization schedule, the optimal dose for maximal immunogenic response is 50 mg total nanoparticle (either 50 mg for one antigen, or 25 mg for two antigens). However, the present invention is not limited to this optimal dose.

In an HLA-A2 transgenic mouse model, the present invention has demonstrated that immunization with nanoparticle formulations containing CpG and CT antigens resulted in a significantly higher specific IFN-γ frequencies compared to unbound antigen and CpG, and an elevated lysis activity towards a target cancer cell line (A375). Additionally, simultaneous delivery of CpG-NYESO-E2 and CpG-MAGE-E2 nanoparticles preserved the effects of the individual antigen nanoparticles, and resulted in an additive IFN-γ secretion and lysis activity relative to each separate nanoparticle formulation. Overall, the results show that co-delivery of a multi-epitope nanoparticle vaccine can elicit higher cell-mediated immune responses than single-epitope formulations. The data show that (1) co-immunization of the E2 nanoparticle vaccines can preserve the individual cell-mediated effects against cancer-testis antigens, and (2) these IFN-γ and lytic effects are additive. In further embodiments, using T helper epitopes within the present nanoparticle vaccines could be another important approach to examine towards increasing efficacy. Altogether, this experiment shows the advantages of using the E2 nanoparticle as an effective vaccine platform to deliver cancer-testis antigens for higher cell-mediated activation.

### Example 2: Combination of PD-1 Treatment and CpG-Gp-E2 Immunization

Combination therapy with anti-PD-1 treatment with the gp100-CpG-E2 nanoparticle vaccine was examined and found to significantly increase survival time and prevent tumor development under pre-existing tumor condition. Referring to FIG. 7, the consolidated data of 2 independent experiments for the vaccine+checkpoint blockade inhibitor combination therapy demonstrated 50% remission of tumors in a particularly aggressive tumor model. Twenty C57BL/6 mice per experiment were inoculated with 10⁴ aggressive B16-F10 cells S.C. at the right flank and were subsequently treated with nanoparticle alone (CpG-gp100-E2), anti-PD1 alone, combined, or PBS (control). No obvious adverse effects in mice were observed, as determined by weight loss, hair loss, and general behavior.

Furthermore, tumor re-challenge of surviving combined-treatment (anti-PD-1+E2 vaccine) mice shows evidence of T cell memory. Any mice that did not develop tumors after 60 days were re-challenged with B16-F10 cells, with no additional treatments. Referring to FIG. 8, mice that survived the combination study until day 60 were re-inoculated on day 64 with 10⁴ B16-F10 (S.C. right flank). Mice were monitored for 40 days or until the tumor reached 1.5 cm in diameter when the animals were euthanized. For the combined-treatment group, it was observed that one of the three mice did not develop tumor (the experiment was terminated 40 days post challenge) while the remaining mice had delayed tumor outgrowth by 7-9 days relative to age-matched animals having received no prior treatment, supporting the presence of T cell memory. In both experiments, the results show that the combination of PD-1 and CpG-gp-E2 nanoparticle significantly increased the survival time of animals compared to each treatment separately, and even without a “boost” immunization. Ultimately, this invention can potentially increase cancer treatment efficacy and provide a new therapeutic strategy for cancer states that are conventionally difficult to treat.

### Example 3: Preventative Treatment of Human Patient with Combination Therapy

The following example describes preventative treatment strategies for a pre-cancerous, tumor-free individual involving a treatment method of embodiments of the present invention.

A 55 year old human female patient presents with germline genetic factors indicating a predisposition for one or more cancers. She visits a physician and undergoes additional genetic testing. She is informed that she has a high probability of developing cancerous tumors. The mutation is poorly expressing in the presence of the normal allele and thus, is a viable “neo-antigen” target for tumor cells that have allelic inactivation or loss and increased expression of the mutant form. The physician recommends preventative treatment with a combination therapy of a non-viral nanoparticle vaccine and an immune checkpoint inhibitor. The patient is prescribed immunizing injections of the nanoparticle vaccine with the “neo-antigen” as the target peptide, combined with administration of immune checkpoint inhibitor therapy. With the elicited immune response, the patient does not develop cancerous tumors at the rate established for populations carrying said germline mutation. None or limited side effects are reported.

### Example 4: Preventative Treatment of Human Patient with Combination Therapy

The following example describes preventative treatment strategies for a pre-cancerous, tumor-free individual involving a treatment method of embodiments of the present invention.

A middle aged individual is having peripheral blood analyzed for tumor associated circulating cell free DNA (cfDNA) and an early biomarker for development of cancer. This individual is informed that analysis of their cfDNA reveals a fusion sequence which yields a neo-antigen peptide that my analytic algorithms is predicted to bind to their one or more of their MHC class I molecules. The managing physician recommends a prophylactic immunization to augment anti-tumor immune surveillance. The patient is prescribed immunizing injections of the nanoparticle vaccine with the “neo-antigen” as the target peptide, combined with administration of immune checkpoint inhibitor therapy. With the elicited immune response, the patient does not develop cancerous tumors and the mutation is cleared from the circulating cfDNA indicting the loss of cells with this mutation.

### Example 5: Cancer Treatment of Human Patient with Combination Therapy

The following example describes treatment strategies for a cancerous individual involving a treatment method of embodiments of the present invention.

A 68 year old human female patient presents with rectal bleeding, anemia, and constant fatigue. She visits a physician and undergoes a fecal occult blood test. She is diagnosed with colon cancer and is found to have a 3.5 cm in diameter tumor on her colon. The patient has a colon resection, which revealed lymph node involvement with the tumor. The physician recommends adjuvant treatment with a non-viral nanoparticle vaccine targeting known TAA and checkpoint inhibitor therapy. The patient remains without evidence of progression or recurrence. No side effects are reported.

### Example 6: Tumor Treatment of Human Patient with CT Antigen Vaccine

The following example describes preventative treatment strategies for a cancerous individual involving a treatment method of embodiments of the present invention.

A 65 year old patient presents with an abnormal chest radiograph that led to a chest CT and ultimately diagnosis of pulmonary adenocarcinoma, with the ALK-EML4 fusion mutation. The patient is initially treated with molecularly targeted therapy but progresses after two years. The patient is then treated with immune checkpoint inhibition. She progresses again two years later. As an alternative to standard chemotherapy the patient is offered combined treatment with a non-viral nanoparticle vaccine targeting the ALK-EML4 fusion protein and checkpoint inhibitor therapy. The patient experiences shrinkage of the lung mass, which stabilizes for two additional years. No additional side effects are reported.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. An immunotherapy composition for treating cancer, comprising a non-viral nanoparticle vaccine and one or more immune checkpoint inhibitors, wherein the non-viral nanoparticle vaccine comprises one or more antigens bound to a nanoparticle.

2. The composition of claim 1, wherein the one or more antigens are bound to an external surface of the nanoparticle.

3. The composition of claim 1, wherein the one or more antigens are human tumor-associated antigens (TAAs).

4. The composition of claim 3, wherein the TAAs comprise cancer-testis antigens.

5. The composition of claim 3, wherein the TAAs comprise gp70 epitopes, gp100 epitopes, NY-ESO-1 epitopes, or MAGE-A3 epitopes, or a combination thereof.

6. The composition of claim 1, wherein the one or more checkpoint inhibitors are inhibitors of PD-1, PD-L1, CTLA-4, B7-X, TIGIT, LAG-3, or TIM-3.

7. An immunotherapy composition comprising a non-viral nanoparticle vaccine comprising one or more human cancer-testis (CT) antigens bound to a nanoparticle.

8. The composition of claim 7, wherein the nanoparticle is an E2 subunit of a pyruvate dehydrogenase complex.

9. The composition of claim 7, wherein the one or more CT antigens are bound to an external surface of the nanoparticle.

10. The composition of claim 7, wherein the one or more CT antigens comprise epitopes of MAGE-A(1, 3, or 4), MAGE-C1, NY-ESO-1, CT-7, CT-10, CT-83, SSX-1, SSX-2, SSX-4, SCP-1, PRAME, or a combination thereof.

11. The composition of claim 7, wherein the nanoparticle vaccine further comprises an adjuvant disposed within the nanoparticle.

12. The composition of claim 11, wherein the antigens and adjuvants are conjugated to the nanoparticle via linkers that are responsive to an acidic and reducing environment, respectively, of an endosome after dendritic cell uptake.

13. The composition of claim 7, wherein the nanoparticle vaccine elicits antigen-specific recognition.

14. A method of treating cancer in a human subject in need thereof, comprising administering to the subject a therapeutically effective amount of an immunotherapy composition comprising a non-viral nanoparticle vaccine comprising one or more antigens bound to a nanoparticle, wherein administration of the immunotherapy composition elicits antigen-specific recognition.

15. The method of claim 14, wherein the immunotherapy composition additionally comprises one or more immune checkpoint inhibitors, wherein administration of the immunotherapy composition elicits antigen-specific recognition while simultaneously blocking checkpoints to remove immune suppression.

16. The method of claim 15, wherein the nanoparticle vaccine is administered separately or in conjunction with the immune checkpoint inhibitors.

17. The method of claim 15, wherein the one or more immune checkpoint inhibitors are inhibitors of PD-1, PD-L1, CTLA-4, B7-X, TIGIT, LAG-3, or TIM-3.

18. The method of claim 14, wherein the one or more antigens are human tumor-associated antigens (TAAs).

19. The method of claim 18, wherein the TAAs comprise cancer-testis antigens.

20. The method of claim 18, wherein the TAAs comprise gp70 epitopes, gp100 epitopes, MART-1, TRP2, NY-ESO-1 epitopes, or MAGE-A3 epitopes, or a combination thereof.
